(19) United States
(12) Patent Application Publication
(10) Pub. No.: US 2025/0072950 A1
(43) Pub. Date: Mar. 6, 2025

(54) Closed Loop High Pressure Generating Cryoablation System

(71) Applicant: Adagio Medical, Inc., Laguna Hills, CA (US)

(72) Inventors: Pedram Pourfard, Rancho Santa Margarita, CA (US); Christopher Massengale, Mission Viejo, CA (US); Alexei V. Babkin, Dena Point, CA (US)

(21) Appl. No.: 18/821,890

(22) Filed: Aug. 30, 2024

Related U.S. Application Data
(60) Provisional application No. 63/580,426, filed on Sep. 4, 2023.

(51) Int. Cl. A61B 18/02; A61B 18/00

(52) U.S. Cl. CPC A61B 18/02; A61B 2018/00077; A61B 2018/00095; A61B 2018/00101; A61B 2018/00577; A61B 2018/00648; A61B 2018/00714; A61B 2018/00863; A61B 2018/0212; A61B 2018/0262

(57) ABSTRACT

A closed loop high pressure generating cryoablation system circulates a working fluid through a catheter to freeze a target tissue. The system includes a plurality of functional cycles or circuits. A freeze circuit is operable to pressurize, cool and drive fluid through the catheter. A refill circuit replenishes fluid from a main reservoir. A pressure overflow circuit prevents over pressurization. Related methods are also described.

### CROSS-REFERENCE TO RELATED APPLICATIONS

This claims priority to provisional patent application No. 63/580,426, filed Sep. 4, 2023, entitled “Closed Loop High Pressure Generating Cryoablation System” the entirety of which is incorporated herein by reference for all purposes.

### FIELD AND BACKGROUND OF THE INVENTION

This is generally directed to surgical apparatuses for applying thermal energy to tissue, and more particularly, to surgical apparatuses operable to ablate tissue by cooling the tissue.

Cryoablation has a wide range of applications including the treatment of heart disease. With reference to FIG. 1, a cryoablation catheter 20 is connected to a cryoablation console 10. The console 10 is shown including a high pressure nitrogen gas (N2) source 12 and a heat exchanger assembly 14. The heat exchanger assembly 14 cools the N2 from the N2 source to the desired temperature for cryoablation.

In the prior art arrangement shown in FIG. 1, the heat exchanger assembly 14 includes a container 30 (e.g., a Dewar) filled with cold liquid nitrogen (LN2) 32, and a heat exchanger 34 submerged in the LN2 which transfers heat from the incoming warm nitrogen gas 40 to the cold coolant 32.

During a cryoablation cycle, the LN2 warms and evaporates to gas 36 as the working fluid (N2) is cooled. Evaporated N2 and return gas from catheter 20 leave the system through exhaust line 46. The longer a case, the more LN2 is evaporated. If too much LN2 evaporates, the heat exchanger assembly cannot sufficiently cool the working fluid for the catheter 20. This is a particular problem in ventricular tachycardia (VT) cases in which freeze cycles can last anywhere from 30 to 65 minutes (or more).

A technique to mitigate the above-described challenge is to refill the Dewar 30 during the middle of the case. To do so, however, the catheter 20 must be disconnected from the patient and the console 10. Then, the reservoir 30 must be refilled. The catheter 20 must be connected and calibration testing must be performed before the procedure can be recommenced. This refilling process can delay the case by a half hour or more. This is undesirable.

Additionally, for long freezing cycles, the nitrogen gas source 12 must be replaced with a new tank once the pressure drops below a limit. The high pressure tanks are heavy, bulky and inconvenient to manage.

Accordingly, a cryoablation apparatus and method that can overcome the above-mentioned challenges is desirable.

### Objects and Advantages of the Invention

An object of the invention is to create a cryoablation delivery system that does not require replenishing the Dewar with external liquid coolant.

An object of the invention is to eliminate the need for external heavy nitrogen tanks.

An object of the invention is to create a cryoablation delivery system that does not require external high pressure gas.

An object of the invention is to continuously generate high pressure coolant (e.g., N2 gas) by boiling liquid coolant (e.g., LN2).

An object of the invention is to eliminate the need for external LN2 containers by condensing Nitrogen gas into liquid Nitrogen within a main reservoir.

An object of the invention is to continuously provide high pressure, cold nitrogen to a cryoablation catheter.

### SUMMARY OF THE INVENTION

A cryoablation system operable with a cryoablation catheter comprises a freeze circuit for cooling and driving fluid through the catheter. The freeze circuit comprises a catheter inlet line for transporting fluid from a first high pressure-generating tank assembly, through a first heat exchanger, through the catheter, and to a first fluid reservoir. A refill circuit replenishes the first high pressure-generating tank assembly with fluid from the first reservoir. The refill circuit comprises a refill line to fluidly connect the first fluid reservoir with the first high pressure-generating tank assembly.

In embodiments of the invention, the first fluid reservoir recondenses the gas from the catheter back into liquid (e.g., LN2), thereby, in a sense, regenerating the working fluid for future use.

In embodiments of the invention, the first reservoir is maintained at a low pressure between 0 and 25 psi, and the first high pressure-generating tank assembly is operable to raise the fluid to at least 1000 psi, and optionally to at least 1500 psi.

In embodiments of the invention, the first high pressure-generating tank comprises a vessel; a heater arranged within the vessel; and a tank body enclosing the vessel, and defining a space between the body and the vessel.

In embodiments of the invention, the first end of the tank body is sealed from fluid in the first reservoir by an O-ring, optionally, a spring seal or indium O-ring.

In embodiments of the invention, the vessel is made of stainless steel, or optionally carbon fiber.

In embodiments of the invention, the first high pressure tank assembly further comprises a pump, optionally a Getter-type pump to evacuate said space.

In embodiments of the invention, the system further comprises a pressure overflow circuit. The pressure overflow circuit includes a pressure relief valve operable to open if the pressure within the vessel exceeds a threshold pressure, and to circulate fluid from the vessel along a pressure relief flowpath through a second heat exchanger, through the vessel, and to the first reservoir, thereby cooling (and limiting pressure buildup of) the fluid in the vessel.

In embodiments of the invention, the flowpath through the vessel comprises a spiral-shaped or coil heat transfer element surrounding the heater.

In embodiments of the invention, the system further comprises a second high-pressure generating tank assembly fluidly connected to the catheter and the first reservoir.

In embodiments of the invention, the freeze circuit comprises at least one cold valve comprising: an inlet; an outlet; a seal surface; a seat adapted to interface with the seal surface; a stem coupled to the seat for moving the seat relative to the seal surface; a housing defining a chamber for the stem to be moved; and an actuator to move the stem; wherein the valve is adapted to withstand cryogenic temperatures below −140, and optionally below −180 C, based on the shape, material and arrangement of the sealing surface, seat, stem and housing.

In embodiments of the invention, the seal comprises an O-ring, and optionally, a spring-seal type of O-ring.

In embodiments of the invention, the seat is machined PCTFE.

In embodiments of the invention, the sealing surface is a high polished metal.

In embodiments of the invention, the actuator is a stepper motor.

In embodiments of the invention, the shape of the stem and housing define a Thermal Bridge Number (Th) ranging from 10 to 32, and optionally from 10 to 18.

In embodiments of the invention, a computer is programmed and operable to control the valves based on measured catheter pressure and flowrate.

In embodiments of the invention, the computer is further programmed and operable to control the heater based on measured catheter pressure and flowrate.

In embodiments of the invention, the high-pressure tank assembly and valves are isolated by vacuum.

In embodiments of the invention, the valves are electronically driven, and automatically adjust pressure and flow through the catheter via a computer feedback loop to ensure optimal catheter performance.

In embodiments of the invention, a cryocooler is operable to cool N2 gas to −196 C to generate liquid nitrogen. In embodiments, the returned gas from the catheter is still cold (−176 C), and only a limited amount of energy is required to recondense the gas back into a liquid in the main reservoir.

In embodiments of the invention, a self-contained system does not rely on the use of external fuel sources, such as gaseous and liquid nitrogen, in order to provide the catheter high pressure cold LN2 sufficient for cryoablation.

In embodiments of the invention, the fluid is nitrogen gas or liquid nitrogen, and optionally near critical nitrogen.

### BRIEF DESCRIPTION OF DRAWINGS

FIG. 1 illustrates a schematic drawing of a prior art cryoablation unit and catheter;

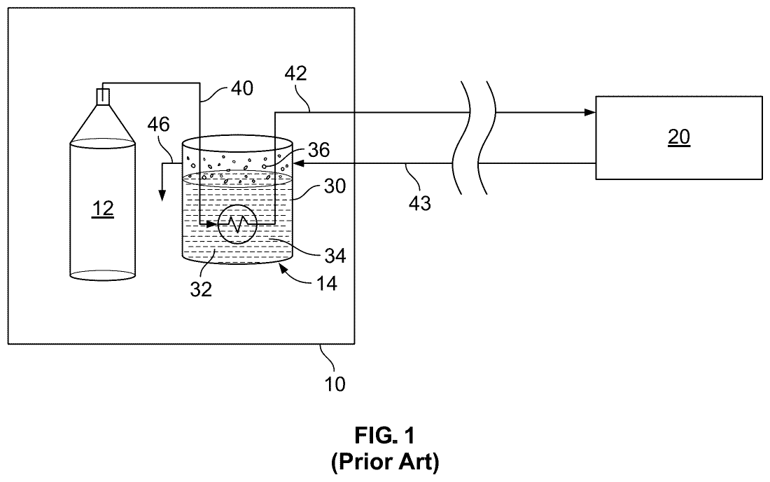

FIG. 2 illustrates a schematic drawing of a cryoablation system including a console and catheter, according to embodiments of the invention;

FIG. 3 is a circuit diagram of various functional circuits of a cryoablation system, according to embodiments of the invention.

FIG. 4A is a front view of a closed loop system, according to embodiments of the invention;

FIG. 4B is a top view of the closed loop system shown in FIG. 4A with the cover removed;

FIG. 5 is a cross sectional view of the closed loop system shown in FIG. 4B taken along line 5-5;

FIG. 6 is a cross sectional view of the closed loop system shown in FIG. 4B taken along line 6-6;

FIG. 7 is a cross sectional view of a high pressure tank assembly, according to embodiments of the invention; and

FIG. 8 is an enlarged cross-sectional view of a cold valve, according to embodiments of the invention.

The description, objects and advantages of embodiments of the present invention will become apparent from the detailed description to follow, together with the accompanying drawings.

### DETAILED DESCRIPTION OF THE INVENTION

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

All existing subject matter mentioned herein (e.g., publications, patents, patent applications and hardware) is incorporated by reference herein in its entirety except insofar as the subject matter may conflict with that of the present invention (in which case what is present herein shall prevail).

Described herein are systems for performing thermal ablation. The systems generally comprise a console and a thermal ablation implement (such as a cryoablation catheter) connected to the console. In a preferred embodiment, the implement is an endovascular cryoablation catheter for treating ventricular tachycardia (VT) such as the catheter described in US Patent Publication No. 20220071680 to Pham et al., filed Jul. 27, 2022, the entire contents of which are incorporated herein by reference for all purposes.

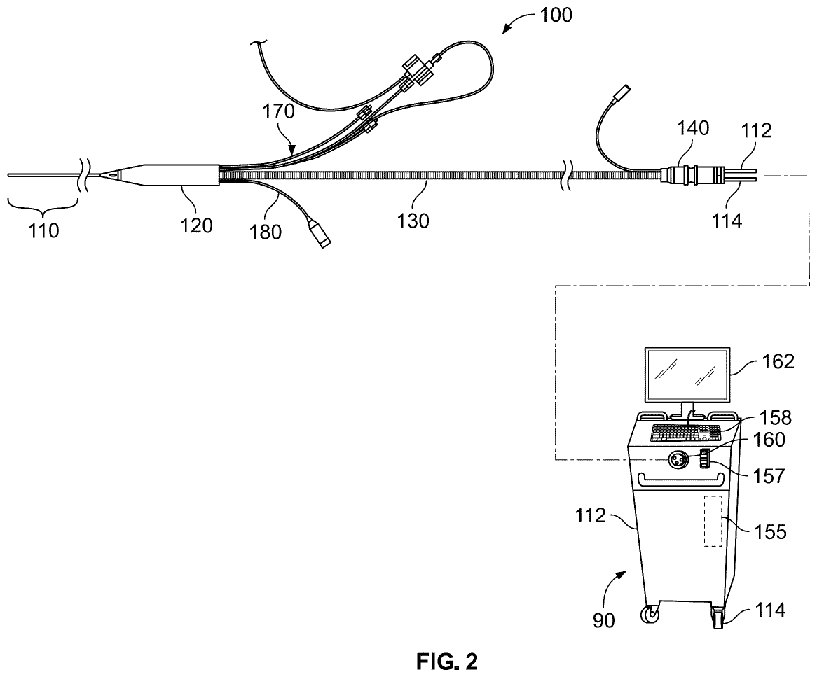

Now with reference to FIG. 2, a cryoablation system in accordance with an embodiment of the invention is described. The cryoablation system is shown including a console 90 and a cryoablation catheter 100 connected thereto. A working liquid such as liquid nitrogen (LN2) is circulated through the catheter via catheter supply line 112 and catheter exhaust line 114. During a case, as the working fluid is circulated through the catheter, heat from the target tissue is transferred to the working fluid. The heat causes the fluid to warm and the tissue to cool.

In embodiments, the temperature of the working fluid along the supply line 112 ranges from −175 to −195 degrees C., and in some embodiments, ranges from −180 to −190 degrees C. In embodiments, the temperature of the working fluid along the exhaust line 114 ranges from −160 to −190 degrees C., and in some embodiments, ranges from −170 to −180 degrees C. In embodiments, the difference in temperature between the catheter supply line 112 and catheter exhaust line 114 ranges from 1 to 20 degrees C., and in some embodiments, from 5 to 15 degrees C.

The cryoablation catheter 100 includes a distal treatment section 110, a handle 120, and an umbilical cord 130. The catheter supply and exhaust lines are arranged within the umbilical cord. The proximal end of the umbilical cord terminates in connector 140, which is inserted into receptacle port 160 on console 90.

One or more ancillary connector lines 170 are shown extending proximally from the handle 120. The tubular lines 170 may serve to provide various functionality including without limitation (a) flushing; (b) vacuum; (c) thermally conductive fluid supply; and/or (d) temperature and pressure sensor conductors.

The catheter 100 is also shown having electrical connector 180 extending proximally from the handle. Electrical connector 180 may be coupled to an EP recording system for analyzing electrical information detected in the distal treatment section 110. Examples of systems for analyzing the electrical activity include, without limitation, the GE Healthcare CardioLab II EP Recording System, manufactured by GE Healthcare, USA and the LabSystem PRO EP Recording System manufactured by Boston Scientific Inc. (Marlborough, MA). The recorded electrical activity may also be used to evaluate or verify the continuous contact with the target tissue as described in U.S. Pat. No. 11,051,867, entitled “TISSUE CONTACT VERIFICATION SYSTEM”, filed Jun. 13, 2018 by Babkin, et al., the entire contents of which are incorporated herein by reference for all purposes.

In addition to the components described above in connection with FIG. 2, console 90 can include a housing 112 and rollers 114 (e.g., castors or wheels). A computer 155 can be arranged within the housing. In embodiments, the computer 155 is programmed and operable to control the components described herein. The console is shown having a built-in dashboard 157 which can include buttons and lights to control and indicate power on/off, mode, time, time elapsed, temperature, etc. The console is also shown including a keyboard 158 and display 162. Optionally, the console includes a communication module and one or more ports to connect directly to external devices including laptops, computers, landlines, and local area networks. Optionally, the console includes a wireless communication module to communicate with portable computer devices including tablets, smart phones and customized devices, catheters, and instruments having wireless capability.

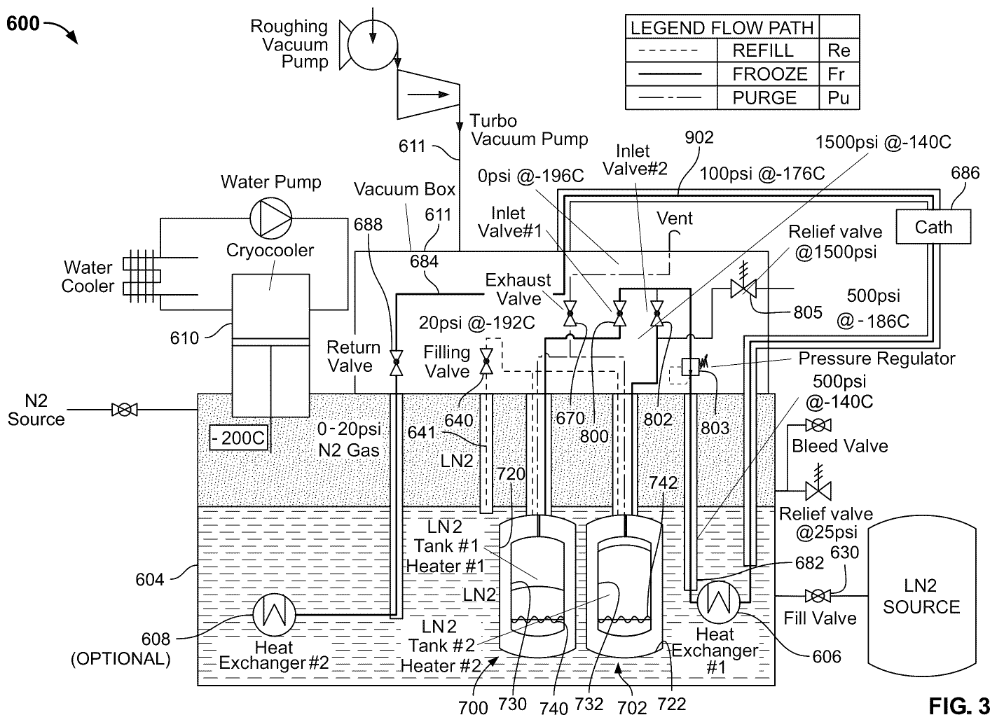

FIG. 3 is a circuit diagram of a cryoablation system 600 according to embodiments of the invention. The cryoablation system 600 shown in FIG. 3 shows, amongst other things, a freeze circuit (Fr), refill circuit (Re), and purge circuit (Pu).

### Freeze or Ablation Circuit

The freeze circuit (Fr) serves to ablate the tissue by circulating high pressure cold fluid through the catheter 686. The freeze circuit comprises a high pressure fluid generator such as first tank assembly 700 (or second tank assembly 702), catheter inlet line 682, main heat exchanger 606, catheter 686, catheter outlet line 684, and main reservoir 604.

### Tank Assemblies

As described further herein, tank assemblies 700, 702 are operable to generate high pressure gas. Each tank assembly 700, 702 includes a heater 740, 742 to boil the liquid nitrogen inside the pressure tank 730, 732. In embodiments, the heaters are controlled to build and maintain pressure to above 500 PSI. The high-pressure tank assemblies 700, 702 are connected to a valve network including a plurality of valves 800, 802 which drives the catheter fluid to the cryoablation delivery catheter 686 and back (via return valve 688) into the main reservoir 604.

### Heat Exchanger

The primary heat exchanger 606 is operable to cool the high pressure gas to the desired cryoablation temperature. The primary heat exchanger 606 is shown submerged in the coolant in the main reservoir 604. Heat is transferred from the fluid in the inlet line 682 to the reservoir coolant via the primary heat exchanger 606 by thermal conduction. An example of a suitable primary heat exchanger is a coil-type heat exchanger. The cooled working fluid then continues along catheter inlet line 682 to catheter 686.

Additionally, in the freeze circuit shown in FIG. 3, a regulator 803 is present along the catheter inlet line 682 to maintain a target pressure (e.g., without limitation 500 psi). Pressure release valve 805 is also arranged along the flow path to release the fluid when the pressure is above a threshold (e.g., above 1500 psi).

The cold fluid departs the catheter and is directed back to the main reservoir 604 via outlet line 684. The returned catheter fluid is combined with the fluid or gas in the main reservoir 604 for reuse, described herein.

Optionally, a second heat exchanger 608 is arranged along the outlet line 684 within the main reservoir to condense the catheter fluid to liquid prior to being released to the main reservoir 604.

### Refill Circuit

The refill circuit (Re) serves to replenish the tank assemblies 700, 702 with coolant from the main reservoir 604. The main reservoir 604 is initially provided or installed with N2 and/or LN2. Preferably, the main reservoir is initially filled by, e.g., a low pressure nitrogen source typically available at a medical suite or hospital. In embodiments, a compressor and filter can be arranged within the system to extract high purity N2 from air as is known to those of skill in the art. Whatever the initial source of nitrogen gas (N2), the N2 is compressed by cryocooler 610 into liquid nitrogen (LN2) inside the main reservoir 604 (e.g., a large Dewar). An exemplary cryocooler is RMS 10T Refrigerator Unit and SA112-C Compressor Unit, ULVAC, Japan.

Once enough liquid nitrogen is generated, it can be transferred to the high pressure tank assemblies 700, 702 via refill network including line 641. After the tank assemblies 700, 702 are filled, the tank assemblies are sealed from the main liquid nitrogen container 604 by fill valve 640. The tank assemblies are now ready to use in the freeze cycle described above.

The reservoir 604 is refilled from the spent catheter fluid transported through the catheter during the freeze cycle. The refill cycle is repeated to fill the tank assemblies 700, 702. In this manner, the LN2 can be continuously recycled and repressurized for use in subsequent ablation freeze cycles.

### Purge Circuit

The purge circuit (Pu) serves to purge or vent the LN2 from the tank assemblies 700, 702. Purge circuit is shown including line 902 and valve 904 to release the N2 through a vent. In embodiments, purging the system is performed to partially bleed excess pressure or completely drain all N2 gas in the Tank. This Purge valve is normally open, and preferably it will be open when there is no power in the system to avoid pressure build up.

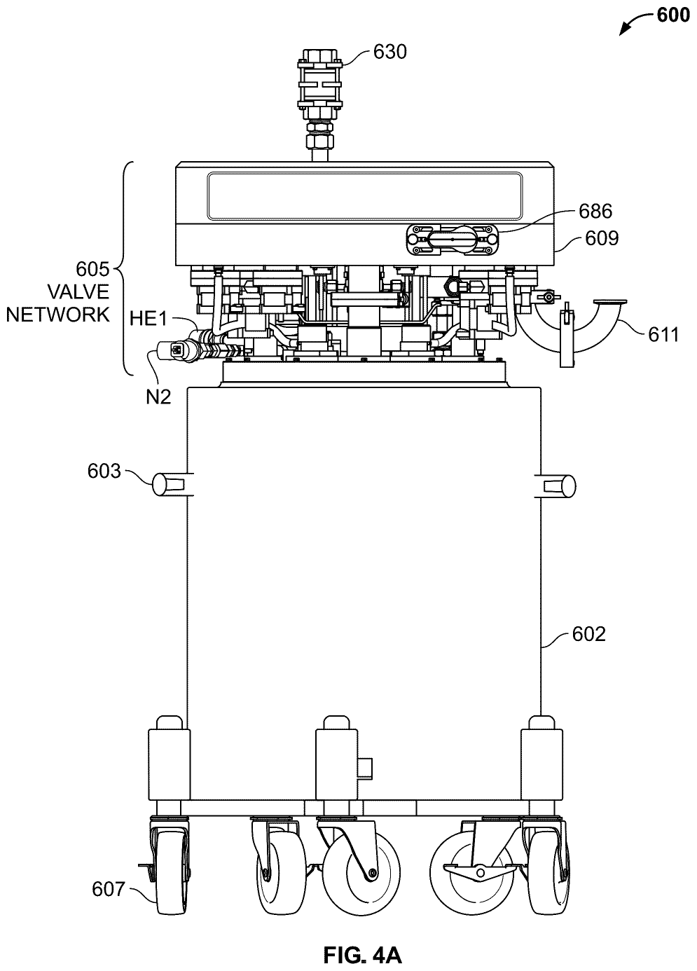

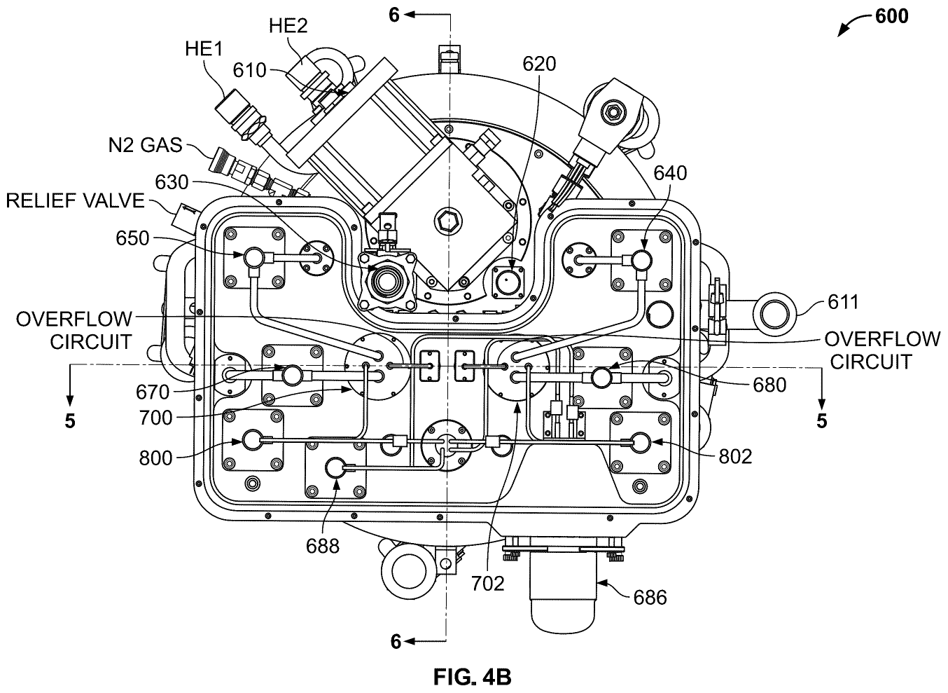

FIGS. 4A, 4B, 5, 6 illustrate various detailed views of a closed loop cryoablation system 600 in accordance with embodiments of the invention.

With reference to FIG. 4A, the system 600 includes a lower body or enclosure 602 which houses the main reservoir, described herein. A handle 603 extends laterally from each side of the body. The enclosure is supported by castors or wheels 607.

A valve network 605 is arranged towards the upper portion of the system 600. A portion of the valve network is encapsulated by a sealed vacuum box 609. Vacuum can be supplied to the vacuum box and other components, described herein, by the u-shaped vacuum pipe 611 extending laterally from the valve network.

Fill port 630 is shown protruding from the valve network. Fill port 630 allows for manual refilling of the main Dewar with LN2 if desired.

With reference to FIG. 4B, which is a top view of the system 600 shown in FIG. 4A with the top of the vacuum box 609 removed for facilitating understanding of the invention, catheter connector 686 is shown on the front of the valve network. This serves to attach the cryoablation catheter to the system for circulating the cold fluid through the catheter for causing tissue ablation.

Additional ports are shown extending from the console including N2 port, first (He1) port, and second (He2) port. The N2 port allows for N2 gas to be added if desired. The (He) ports allow for helium to be added and removed from the cryocooler, described herein.

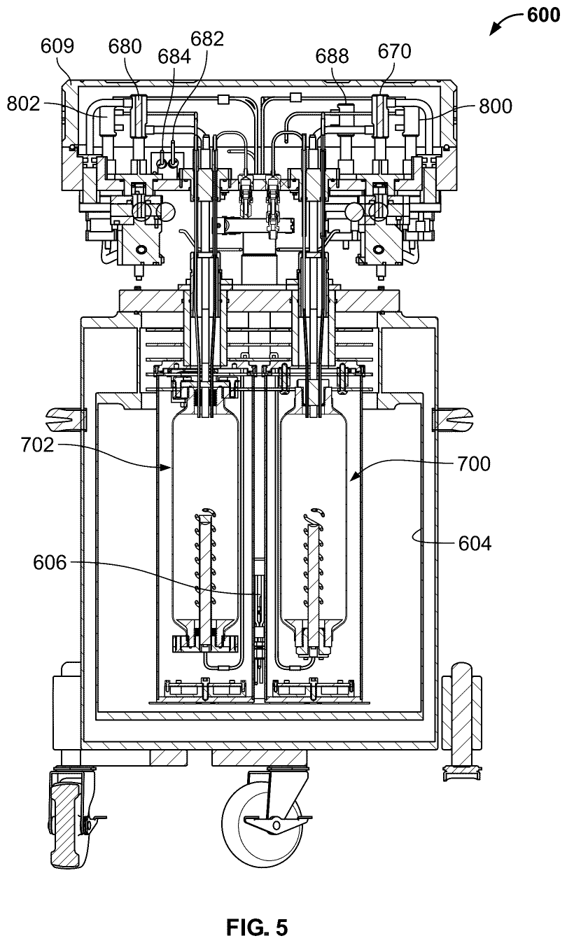

With reference to FIG. 5, (which is a cross-sectional view of the system 600 shown in FIG. 4B, taken along line 5-5), high pressure tank assemblies 700, 702 are shown arranged inside the main reservoir 604. The tank assemblies 700, 702, as described herein, are thermally insolated and operable to generate high pressure N2 by heating the N2. Cold inlet valve 800 (or 802) is electronically opened to permit the high pressure N2 to pass from the tank assembly 700 through the first heat exchanger 606, thereby cooling the fluid to a target cryoablation temperature. In embodiments, the target ablation temperature of the fluid ranges from −150 to −200 deg. C, and in some embodiments from −180 to −195 deg. C.

FIG. 5 also shows catheter inlet and outlet lines 682, 684 respectively which connect to the corresponding lumens extending though the ablation catheter via the catheter connector 686 shown in FIGS. 4A, 4B.

Cold return valve 688 is open during the freeze cycle to allow the returning catheter fluid into the main reservoir 604. This valve is closed during catheter removal and Dewar pressurization.

Cold exhaust valve 670, 680 seal the tank assemblies 700, 702 respectively from the environment, allow air to escape in the filling process, and also can be used to rapidly depressurize the tanks in case of incident.

In embodiments, each tank assembly has its own exhaust valve so that each tank may be independently drained, rather than requiring both tanks to be drained at the same time.

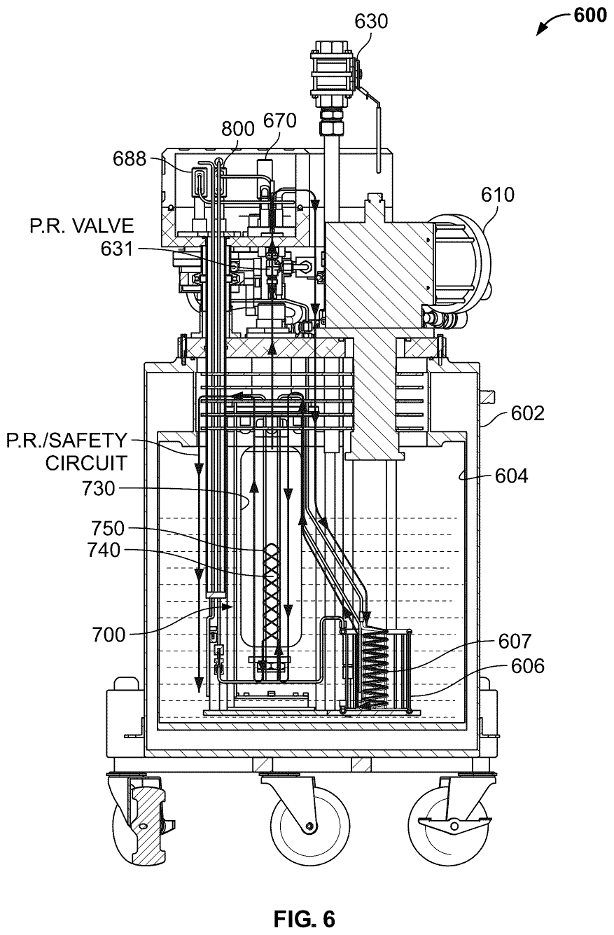

With reference to FIG. 6, which is a cross sectional view of the system 600 shown in FIG. 4B taken along line 6-6, first heat exchanger 606 is shown submerged within the LN2 of the main reservoir 604. Heat exchanger 606 transfers cold to the catheter inlet line 682 from the LN2 maintained in the reservoir 604. First heat exchanger is shown in the shape of a cylinder having straight parallel heat transfer elements running axially along the circumference of the heat exchanger.

A second heat exchanger 607 is shown arranged within the main reservoir and, optionally, within the first heat exchanger 606. Second heat exchanger 607 is shown having a coil or spiral shape. The second heat exchanger 607 transfers cold to the pressure tank assembly to relieve pressure, as described further herein. Arranging the heat exchangers concentrically serves to conserve space, reducing the footprint of the main reservoir.

### Pressure Relief and Safety Circuit

The second heat exchanger 607 is part of a safety circuit to cool the heaters 740,742 in the pressure tank assemblies 700,702 in the event of over pressurization. In embodiments, when the pressure within the circuit reaches an upper safety limit, a pressure relief valve 631 opens permitting the high pressure N2 to flow out the tank, through the second heat exchanger 607, back to the vessel 730, through the overflow coil 750, and ultimately back into the main reservoir 604. As the cool gas flows through overflow coil 750, it cools down the heater 740 and slows pressure build up in the tank 730. The pressure relief or safety circuit serves to mitigate over pressurization. In embodiments, the upper pressure safety limit ranges from 600 psi to 800 psi.

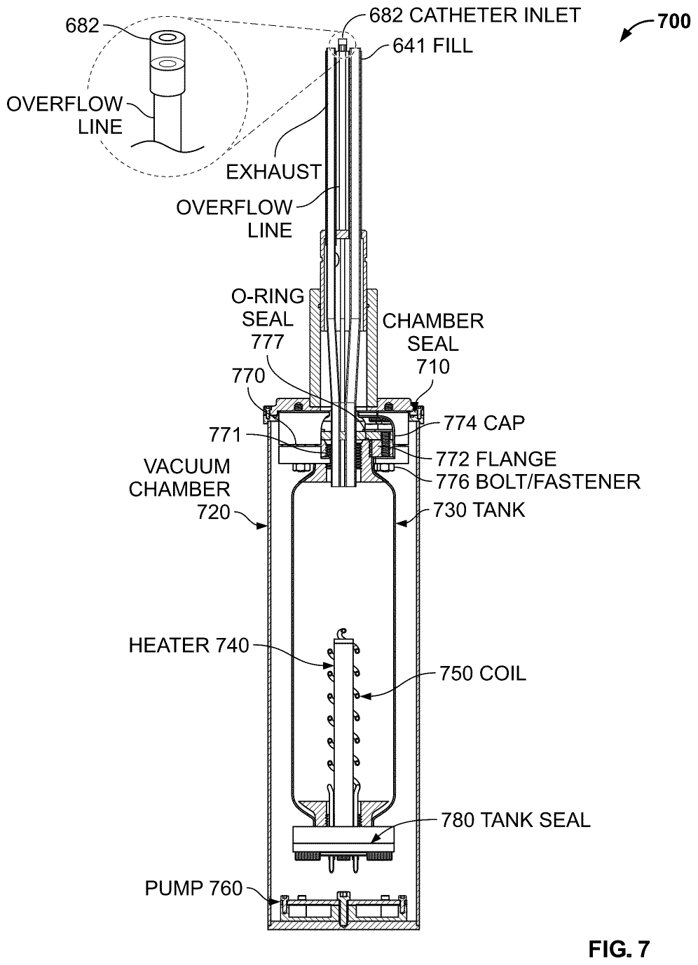

FIG. 7 is an enlarged cross-sectional view of pressure tank assembly 700 in accordance with embodiments of the invention.

Assembly 700 is shown having an outer body 720 with a cylindrical shape. Tank 730 is arranged within the outer body 720. An evacuated space or vacuum is maintained between the tank and body for insulating the heat generated in the tank from the LN2 surrounding the tank assembly (not shown). Although the outer body 720 may be coupled to a vacuum source as described above, in embodiments, a dedicated pump 760 (e.g., a Getter pump) is provided to maintain vacuum within the body. An example of a suitable pump is an activated charcoal vacuum pump, which can automatically pull and maintain vacuum when submerged in liquid nitrogen.

In embodiments, the vessel 730 is made of stainless steel or optionally carbon. The inventors have found carbon is a preferred material because it can withstand high pressure, is light, occupies less volume, and can be custom shaped. The carbon fibers can be glued together to form the vessel. Its ends can be custom formed with external threads. In embodiments, the carbon vessel is made with one or more sensors incorporated in the walls to measure fluid contained within the vessel. Examples of sensors that may be incorporated into the wall of the vessel include temperature and pressure sensors.

A heater 740 is shown arranged within the tank 730. The heater 730 is operable to rapidly build pressure inside the carbon pressure vessel by boiling the liquid nitrogen. The heater 740 can be used actively during the ablation/freeze phase to maintain high pressures while the gas flows through the catheter. In embodiments, the heater is a resistive-type heater, and can run on electricity.

As described above, the high pressure tank assembly comprises a safety circuit to mitigate over pressurization of the tank. For example, in embodiments, a pressure overflow coil 750 surrounds the heater. In the event of rapid over pressurization of the tank, the pressure relief valve (not shown in FIG. 7) opens, and excess gas is delivered from the vessel 730, through a heat exchanger 607, and through the pressure overflow coil 750. The cool gas cools the heater and slows pressure build up in the tank 730.

FIG. 7 also shows a first seal 710 to seal the internal vacuum chamber defined between the body 720 and tank 730 and the external cold main reservoir/Dewar environment 604 (not shown). In preferred embodiments, an indium seal or O-ring is provided between the mating components.

FIG. 7 also shows a second seal 770 to seal the inside of the carbon tank 730 from the surrounding vacuum. In embodiments, the carbon tank 730 includes ends with external threads 771. The tank end is screwed into flange 772 over which a cap 774 is bolted 776 thereto. An O-ring seal, preferably indium seal, is arranged between the cap and tank end to provide an air tight hermetic seal and operable within the cryogenic environment. The indium is compressed between the carbon/epoxy surface of the tank 730 and a high polish metal O-Ring groove in the cap 774. A similar seal 780 is provided at the lower end of the tank.

In embodiments of the invention, the heater 740 is isolated from the high-pressure tank by a cartridge that is welded to the flange. The cartridge serves to prevent the heater from being damaged by high pressures and cold temperatures. The welded cartridge also keeps the high pressure tank 730 from leaking N2 and LN2 into its surrounding vacuum chamber. However, in other embodiments, the heater is not enclosed by a sheath, and the heater is joined directly to the flange.

FIG. 7 also shows four (4) fluid transport lines or conduits extending into the tank 730 including a conduit for the pressure relief circuit (overflow line), a conduit for the catheter freeze circuit 682, a conduit for the refill circuit 641, and a conduit for the tank exhaust. Each conduit is sealed fluid-tight to the tank by welding each tube to the cap 774 and it is thermally isolated by vacuum all the way to tip of the tube.

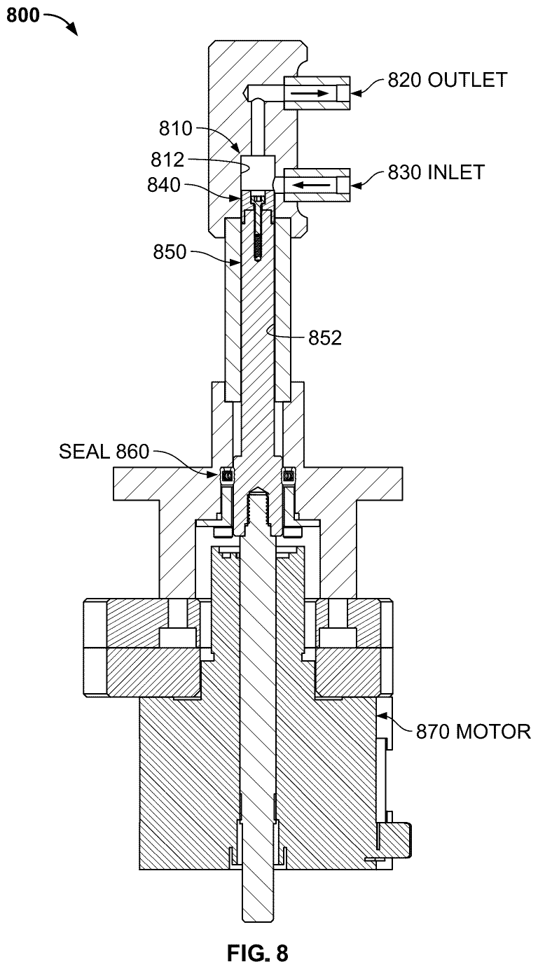

FIG. 8 is an enlarged view of a cold valve 800 in accordance with embodiments of the invention. The valve 800 is operable to permit high pressure fluid from the tank assembly 700 to the catheter inlet line 682 which runs through the first heat exchanger 606 in the main LN2 reservoir and to the catheter. The valve 800 must be fluid and air tight, as well as temperature resistant despite repeated use. The cold valve 800 is used to seal and prevent leaks from the cold, high pressure tank 730. The inventors have designed a piston-like valve, an embodiment of which is shown in FIG. 8 and described herein, to operate under such conditions.

The valve 800 shown in FIG. 8 includes an inlet 830 for the fluid, typically high pressure gas or liquid, to enter and an outlet 820 for gas or liquid to exit when the valve is opened.

The valve 800 shown in FIG. 8 also includes a chamber defining a sealing surface 810. In embodiments, the sealing surface is a high polish metal surface.

Seat 840 is operable to press into the sealing surface 810 with high force and blocks flow through the valve, even at extremely cold temperatures. In embodiments, the seat is made of PCTFE, and machined to shape.

Stem 850 is operable to move the seat 840 to and from the sealing surface, thereby respectively closing and opening the valve. An O-ring 860 is arranged between the stem and the valve body to prevent gas from the inlet from escaping through the bottom of the valve. In embodiments, the O-ring 860 is a Teflon encapsulated spring seal.

In the valve 800 shown in FIG. 8, a stepper motor 870 is operable to actuate the stem 850, imparting significant force on the sealing surface when the valve is closed. Additionally, in embodiments, the motor is operable to precisely control the distance between the sealing surface 810 and the seat 840, thereby controlling the flowrate of the fluid through the valve. In embodiments, a stored database correlates stem location with flowrate. The user can thus instruct the system to run at one flowrate, and the computer instructs the motor to move the stem to the location to achieve the desired flowrate.

In embodiments of the invention, the arrangement of the stem 850 and the valve housing 852 minimizes cooling of the seal 860. Inventors have identified a challenge solved by embodiments of the invention is to prevent the seal 860 from cooling, and thereby shrinking. When the O-ring shrinks, the valve leaks.

The inventors have overcome this challenge by a combination of design parameters including: reducing the gap between the stem and the housing; increasing the surface area of the inner wall of the housing, lengthening the stem, or any combination of same. In embodiments, it is desirable to reduce the thermal bridge between the cold inlet gas (e.g., inlet 830) and the valve seal (e.g., O-ring 860) based on the following:

$$Thermal bridge number ( Th ) ∝ \frac{Gap}{Legnth} ;$$

where the ‘Gap’ is the spacing between the stem and housing, ‘Length’ is the length of the stem. Reducing the thermal bridge number (Th) is desirable to warm the seal (or prevent the seal from shrinking, and leaking). In embodiments, the gap ranges from about 0.05 to 0.13 mm. The stem length ranges from 12.5 to 40 mm. And the Thermal bridge number (Th) ranges from 10 to 32 and is preferably between 10 and 18

It is to be understood that the valves described herein can be identical or similar to valve 800. Additionally, the valves are constructed with the system such that inlet and outlet conduits are removable, and/or the whole valve assembly can be removed as a unit for maintenance or replacement. It is also to be understood that the location of certain features of the valve such as the inlet and outlet, materials, motor, and other components may be arranged differently, modified, or removed where doing so is not exclusive to the function of the valve. For example, the inlet may be arranged on the same or opposite side as the outlet, or the inlet may be arranged above or below the outlet.

### Control

The systems described herein can be connected and controlled by a computer and or programmed processors to open and close the valves, adjust the temperature of the heaters, and to adjust the flowrate or speed of the pumps. The control valves and heaters can be controlled to maintain flowrate based on pressure measurement and or flowrate measurement. A wide range of different types of sensors can be incorporated throughout the circuits to measure pressure, temperatures, flowrate, and liquid level. The sensed data may be evaluated by the computer and an algorithm to: (a) maintain flowrate and pressure of the cold fluid circulated through the catheter; (b) refill the pressure tank assemblies; (c) purge the system; and (d) control the safety overflow circuit as described above.

In embodiments, a user interface provides a display, optionally touchscreen, and keyboard, mouse, or another user interface device permitting an operator to provide instructions to the system. The interface is operable to communicate with the computer to receive the instructions and carry out the functions and circuits described herein. All data may be stored in a memory or storage device and processed to be sent or output to a display or another device.

### Alternative Embodiments

In embodiments, the first heat exchanger (e.g., heat exchanger 606) can be removed depending on the application; e.g., where the gas boiling in the high pressure chamber is already cold (−120 to −160 C depending on pressure) the first heat exchanger can be removed.

In embodiments, the regulator (e.g., pressure regulator 803) can be removed depending on the application; e.g., where the PID is adjusted by monitoring the pressure in the vessel and adjusting the heater power level accordingly.

The consoles described herein may include one or more valves, pumps, fans, temperature and pressure sensors, and regulators along the flowpaths for starting and stopping flow, controlling pressure, controlling flowrates, controlling temperature, emergency stop, and controlling cooling power.

Additionally, although an exemplary cooling liquid is liquid nitrogen (LN2), other coolants may be used and are intended to be included within the scope of the invention except where specifically excluded in any appended claims.

Additionally, although an exemplary apparatus is a catheter, the invention described above can be applied to circulate a cool working fluid through a wide variety of thermal implements including, without limitation, flexible catheters, rigid probes, instruments, and devices.

Throughout the foregoing description, and for the purposes of explanation, numerous specific details are set forth in order to provide a thorough understanding of the described techniques. It will be apparent, however, to one skilled in the art that these techniques can be practiced without some of these specific details. Although various embodiments that incorporate these teachings have been shown and described in detail, those skilled in the art could readily devise many other varied embodiments or mechanisms to incorporate these techniques. Also, embodiments can include various operations as set forth above, fewer operations, or more operations; or operations in another order than that specifically described above. Additionally, any of the components and steps described herein may be combined with one another in any logical manner except where such components or steps would be exclusive to one another. Accordingly, the scope and spirit of the invention should be judged in terms of the claims, which follow as well as the legal equivalents thereof.

1. A cryoablation system operable with a cryoablation catheter, the system comprising:
a freeze circuit for cooling and driving fluid through the catheter, the freeze circuit comprising a catheter inlet line for transporting fluid from a first high pressure-generating tank assembly, through a first heat exchanger, through the catheter, and to a first fluid reservoir, and
a refill circuit to replenish the first high pressure-generating tank assembly with fluid from the first reservoir, the refill circuit comprising a refill line to fluidly connect the first fluid reservoir with the first high pressure-generating tank assembly.

2. The system of claim 1, wherein spent gas from the catheter is recondensed to liquid in the first fluid reservoir; and the fluid in the first reservoir is maintained at a low pressure between 0 and 25 psi.

3. The system of claim 2, wherein the first high pressure-generating tank assembly is operable to raise the fluid to at least 1000 psi.

4. The system of claim 3, wherein the first high pressure-generating tank assembly comprises:
a vessel;
a heater arranged within the vessel; and
a tank body enclosing the vessel, and defining a space between the body and the vessel.

5. The system of claim 4, wherein a first end of the vessel is sealed from the space by an O-ring.

6. The system of claim 4, wherein a first end of the tank body is sealed from fluid in the first reservoir by spring seal or indium type of O-ring.

7. The system of claim 4, wherein the first high pressure-generating tank assembly further comprises a pump to evacuate said space.

8. The system of claim 4, further comprising a pressure overflow circuit, the pressure overflow circuit comprising a pressure relief valve operable to open if the pressure within the vessel exceeds a threshold pressure, and to circulate fluid from the vessel along pressure relief flowpath through a second heat exchanger, through the vessel, and to the first reservoir, thereby cooling the fluid in the vessel.

9. The system of claim 8, wherein the flowpath through the vessel comprises a spiral-shaped (or coil) heat transfer element surrounding the heater.

10. The system of claim 1, further comprising a second high-pressure generating tank assembly fluidly connected to the catheter and the first reservoir.

11. The system of claim 1, wherein the freeze circuit comprises a cold valve comprising:
an inlet;
an outlet;
a seal surface;
a seat adapted to interface with the seal surface;
a stem coupled to the seat for moving the seat relative to the seal surface; and
a housing defining a chamber for the stem to be moved; and
an actuator to move the stem,
wherein the valve is adapted to withstand cryogenic temperatures below −140 based on the shape, material and arrangement of the sealing surface, seat, stem and housing.

12. The system of claim 11, further comprising seal between the stem and the housing, and wherein the seal comprises an O-ring.

13. The system of claim 11, wherein the seat is machined PCTFE.

14. The system of claim 11, wherein the sealing surface is a high polished metal.

15. The system of claim 11, wherein the actuator is a stepper motor.

16. The system of claim 11, wherein the shape of the stem and housing define a Thermal Bridge Number ranging from 10 to 32.

17. The system of claim 8, further comprising a computer programmed and operable to control the valves based on measured catheter pressure or flowrate.

18. The system of claim 1, further comprising a computer programmed and operable to control the heater based on measured catheter pressure or flowrate.

19. A cold valve comprising:
an inlet;
an outlet;
a seal surface;
a seat adapted to interface with the seal surface;
a stem coupled to the seat for moving the seat relative to the seal surface; and
a housing defining a chamber for the stem to be moved; and
an actuator to move the stem,
wherein the valve is adapted to withstand cryogenic temperatures below −140 based on the shape, material and arrangement of the sealing surface, seat, stem and housing, and wherein the shape of the stem and housing define a Thermal Bridge Number ranging from 10 to 32.

20. A method for performing cryoablation on a tissue comprising:
providing a high pressure tank of fluid in a condensed liquid phase;
circulating the fluid in the condensed liquid phase from the high pressure tank, through an ablation apparatus, and into a main reservoir, wherein an amount of the fluid returned to the main reservoir expands to a gas phase;
recondensing the returned fluid in the gas phase to the liquid phase in the main reservoir; and
refilling the high pressure tank of fluid with the recondensed fluid in the liquid phase.
